(19) United States
(12) Patent Application Publication
(10) Pub. No.: US 2024/0268767 A1
(43) Pub. Date: Aug. 15, 2024

(54) METHOD FOR ANALYZING SIGNAL WAVEFORM, ELECTRONIC APPARATUS, AND COMPUTER-READABLE RECORDING MEDIUM

(71) Applicant: Han-Hwa Hu, Taipei City (TW)

(72) Inventors: Han-Hwa Hu, Taipei City (TW); Wen-Yung Sheng, Taipei City (TW); Hung-Yi Hsu, Taichung City (TW); Ke Han, Shenzhen (CN); Cheng-Yen Wang, Taoyuan City (TW)

(73) Assignee: Han-Hwa Hu, Taipei City (TW)

(21) Appl. No.: 18/337,446

(22) Filed: Jun. 20, 2023

(30) Foreign Application Priority Data
Feb. 10, 2023 (TW) 112104835

(51) Int. Cl. A61B 5/00; A61B 5/021; A61B 5/0285

(52) U.S. Cl. CPC A61B 5/7257; A61B 5/021; A61B 5/0285

(57) ABSTRACT

A method for analyzing signal waveform, an electronic apparatus, and a computer-readable recording medium are provided. The method is described below. A physiological signal waveform is obtained. A section waveform is obtained by using a time segment every sampling interval from the physiological signal waveform, and a power ratio of the section waveform within the first frequency range and the second frequency range is calculated. A statistical calculation is performed for multiple power ratios corresponding to multiple section waveforms in multiple time sections obtained from the physiological signal waveform using the time segment. A statistical result of the statistical calculation is output to a user interface.

### CROSS-REFERENCE TO RELATED APPLICATION

This application claims the priority benefit of Taiwan application serial no. 112104835, filed on Feb. 10, 2023. The entirety of the above-mentioned patent application is hereby incorporated by reference herein and made a part of this specification.

### BACKGROUND

### Technical Field

The disclosure relates to a signal analysis technology, and more particularly, to a method for analyzing signal waveform for medical assistance, an electronic apparatus, and a computer-readable recording medium.

### Description of Related Art

In recent years, the global aging trend has become increasingly serious, and among the elderly population, the incidence of dementia (Alzheimer's disease) has increased rapidly. However, the current detection and measurement methods for dementia risk are generally too complicated, which makes them difficult to promote and unable to meet relevant needs.

### SUMMARY

The disclosure provides a method for analyzing signal waveform, an electronic apparatus, and a computer-readable recording medium, which provides effective medical assistance.

The method for analyzing signal waveform of the disclosure is executed by a processor, and the method is described below. A physiological signal waveform is obtained. A section waveform is obtained by using a time segment every sampling interval from the physiological signal waveform, and a power ratio of the section waveform is calculated. The process of calculating the power ratio of the section waveform is described below. The section waveform is converted into a spectrum. A first power sum within a first frequency range of the spectrum is calculated. A second power sum within a second frequency range of the spectrum is calculated. The first frequency range lies within the second frequency range. A ratio of the first power sum to the second power sum is calculated to obtain the power ratio. A statistical calculation is performed for multiple power ratios corresponding to multiple section waveforms in multiple time sections obtained from the physiological signal waveform using the time segment. A statistical result of the statistical calculation is output to a user interface.

In an embodiment of the disclosure, the physiological signal waveform is a cerebrovascular resistance waveform, and the method is further described below. A blood pressure waveform within a measurement time is obtained through a first sensor. A cerebral blood flow velocity waveform within the measurement time is obtained through a second sensor. Multiple blood pressures and multiple cerebral blood flow velocities corresponding to multiple heartbeat cycles are obtained from the blood pressure waveform and the cerebral blood flow velocities waveform to calculate a cerebrovascular resistance value of each of the heartbeat cycles, respectively, and the cerebrovascular resistance waveform is obtained.

In an embodiment of the disclosure, the process of calculating the cerebrovascular resistance value of each of the heartbeat cycles is described below. A blood pressure average value (mean blood pressure) corresponding to the blood pressures of each of the heartbeat cycles is calculated. A velocity average value of the cerebral blood flow velocities (mean cerebral blood flow velocity) corresponding to each of the heartbeat cycles is calculated. The mean blood pressure value is divided by the mean cerebral blood flow velocity value to obtain the cerebrovascular resistance value.

In an embodiment of the disclosure, the first sensor is a blood pressure monitor, and the second sensor is a transcranial Doppler (TCD) ultrasonic device.

In an embodiment of the disclosure, the physiological signal waveform is a cerebral blood flow velocity waveform, and the method is further described below. The cerebral blood flow velocity waveform within a measurement time is obtained through a TCD ultrasonic device.

In an embodiment of the disclosure, the physiological signal waveform is a blood pressure waveform, and the method is further described below. The blood pressure waveform within a measurement time is obtained through a blood pressure monitor (a servo-controlled plethysmograph).

In an embodiment of the disclosure, the statistical calculation includes at least one of the following process: calculating an average value of the power ratios; calculating a coefficient of variation of the power ratios; calculating a first quartile, a second quartile, and a third quartile of the power ratios; and calculating an area occupied by power ratios greater than a default value in a power ratio tendency waveform obtained based on the power ratios.

In an embodiment of the disclosure, the first frequency range is 0.02-0.04 Hz, and the second frequency range is 0.02-0.07 Hz.

An electronic apparatus of the disclosure includes: a storage, storing at least one code segment; and a processor, coupled to the storage, and configured to execute the at least one code segment for implementing the method for analyzing signal waveform.

A non-transitory computer-readable recording medium of the disclosure is configured to store a code. In response to the code being executed by a processor, the processor is made to execute each of the processes of the method for analyzing signal waveform.

Based on the above, the disclosure analyzes the power ratios corresponding to a specified frequency range in the section waveforms of the physiological signal, and performs statistical calculation on the power ratios, then outputs the statistical result to the user interface, so that the user may more intuitively determine whether there is an abnormal risk in a person to be detected.

### BRIEF DESCRIPTION OF THE DRAWINGS

FIG. 1 is a block diagram of an analysis system for analyzing brain-related signals according to an embodiment of the disclosure.

FIG. 2 is a flowchart of a method for analyzing signal waveform according to an embodiment of the disclosure.

FIG. 3 is a schematic diagram of a cerebral blood flow velocity waveform according to an embodiment of the disclosure.

FIG. 4 is a schematic diagram of a spectrum according to an embodiment of the disclosure.

FIG. 5 is a schematic diagram of a power ratio tendency waveform according to an embodiment of the disclosure.

### DESCRIPTION OF THE EMBODIMENTS

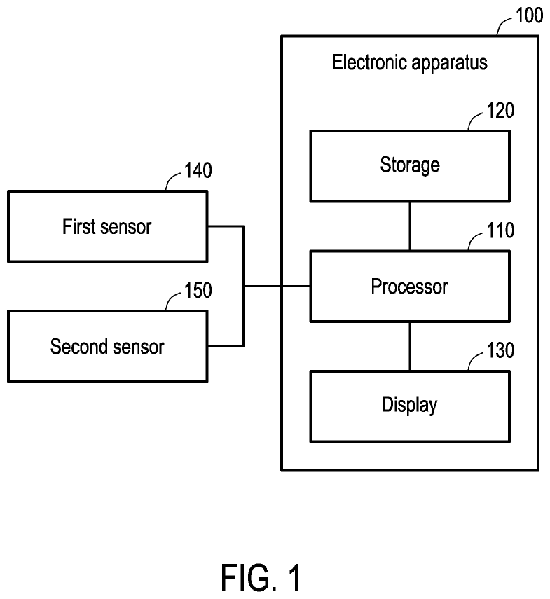

FIG. 1 is a block diagram of an analysis system for analyzing brain-related signals according to an embodiment of the disclosure. Referring to FIG. 1, the analysis system includes an electronic apparatus 100, a first sensor 140, and a second sensor 150. The electronic apparatus 100 includes a processor 110, a storage 120, and a display 130. The processor 110 is coupled to the storage 120, the display 130, the first sensor 140, and the second sensor 150. The first sensor 140 and the second sensor 150 herein are just examples and not limited thereto.

The first sensor 140 is configured to obtain a blood pressure waveform within a measurement time. The first sensor 140 may be realized by using a non-invasive blood pressure monitor. For example, the non-invasive blood pressure monitor is a wrist-worn blood pressure monitor or a finger blood pressure monitor (a servo-controlled plethysmograph). The first sensor 140 is configured to measure the blood pressure wave of the person to be detected within a measurement time, and then obtain the blood pressure waveform. The wave of the blood pressure (mmHg) of the person to be detected within a measurement time (e.g., 5 to 10 minutes) is collected through the non-invasive blood pressure monitor.

For example, in the case of using a finger blood pressure monitor, a height calibrator may be used to calibrate the height difference between the finger wearing the device and the heart before taking the measurement, so as to avoid the influence of the position of the hand on accuracy.

The second sensor 150 may be realized by using a transcranial Doppler (TCD) ultrasonic device. The second sensor 150 obtains a cerebral blood flow velocity waveform within the measurement time. The cerebral blood flow velocity (cm/s) of the middle cerebral artery on the right, left, or both sides within a measurement time are collected through the TCD ultrasonic device, that is, the cerebral blood flow velocity wave, and then the cerebral blood flow velocity waveform is obtained.

In an embodiment, in the case that the TCD ultrasonic device is used as the second sensor 150, a monitoring head frame is put on the person to be detected first before recording the cerebral blood flow velocity through the TCD ultrasonic device. After fixing the probe, the TCD ultrasonic device is set to a dual-channel single-depth mode. The sampling depth is 50-65 millimeters, the sampling bulk is 10-15 cubic millimeters, and the measurement time is 5-10 minutes. The TCD ultrasonic device is used to monitor the middle cerebral artery (MCA) of the bilateral (left and right) brains of the person to be detected. The gain adjustment of the TCD ultrasonic device is preferably smooth with no burr-like changes in the envelope of the blood flow speed spectrum. Through the simultaneous monitoring of the left middle cerebral artery and the right middle cerebral artery of the person to be detected, subsequent processing and analysis are performed on the obtained left mean cerebral blood flow velocity value and right mean cerebral blood flow velocity value of the person to be detected.

The electronic apparatus 100 is a device with a computing function, such as a smart phone, a tablet computer, a notebook computer, a personal computer, and the like. The electronic apparatus 100 includes a processor 110, a storage 120, and a display 130. The processor 110 is coupled to the storage 120 and the display 130.

The processor 110 is, for example, a central processing unit (CPU), a physics processing unit (PPU), a programmable microprocessor, an embedded control chip, a digital signal processor (DSP), an application specific integrated circuits (ASIC), or other similar devices.

The storage 120 is, for example, any type of repaired or removable random access memory (RAM), read-only memory (ROM), flash memory, hard disk, or other similar device or a combination of these devices. The storage 120 includes one or more code segments. After being installed, the code segments are executed by the processor 110 to implement the following method for analyzing signal waveform.

The display 130 is, for example, a liquid crystal display (LCD), a plasma display, and the like.

In some embodiments, detectable physiological signals include blood pressure, heart rate, cerebral blood flow velocity, cerebrovascular resistance value, etc. The cerebrovascular resistance value may be calculated as mean blood pressure divided by mean cerebral blood flow velocity according to Ohm's law. For blood pressure, heart rate, cerebral blood flow velocity, and cerebrovascular resistance value, the transfer function analysis may be used to divide the wave into three frequency ranges: very low frequency range, low frequency range, and high frequency range. Then, the linkage between the relations are analyzed. The high frequency range is related to the respiratory and/or parasympathetic nerve, the low frequency range is related to the sympathetic nerve or vasomotor, and the very low frequency range is related to the brain tissue or brain pressure.

In other embodiments, factors affecting the detection environment, such as ambient temperature, measuring time section, and ambient volume, may be further limited, and physiological factors such as diet and sleep of the person to be detected may also be limited. For example, the detection environment is set in a space with an air conditioner, and the air conditioner is used to control the ambient temperature at 22-24° C. In addition, due to changes in the circadian rhythm, detection is limited to similar time sections to ensure reproducibility. In addition, the stimulation of vision or hearing (including the interference of people entering and leaving) to the person to be detected is reduced. For a period of time (e.g., 12 hours) before the measurement, the person to be detected is restricted from consuming caffeinated beverages, chocolate, and indigestible food. In addition, avoid exercise, alcohol intake, and consuming food or drugs that may affect the analysis results at least 12 hours before the test. In addition, the person to be detected should first rest for 15 minutes (to ensure that the blood pressure, heart rate, and heartbeat are stable) and then take a supine position (the position of the head is recorded at the same time) or a sitting position (without crossing the lower limbs) for detection.

When collecting the blood pressure wave and the cerebral blood flow velocity wave of the person to be detected, the recording is continued for at least 5 minutes. Generally, at least 10 minutes of blood pressure and cerebral blood flow velocity data are required.

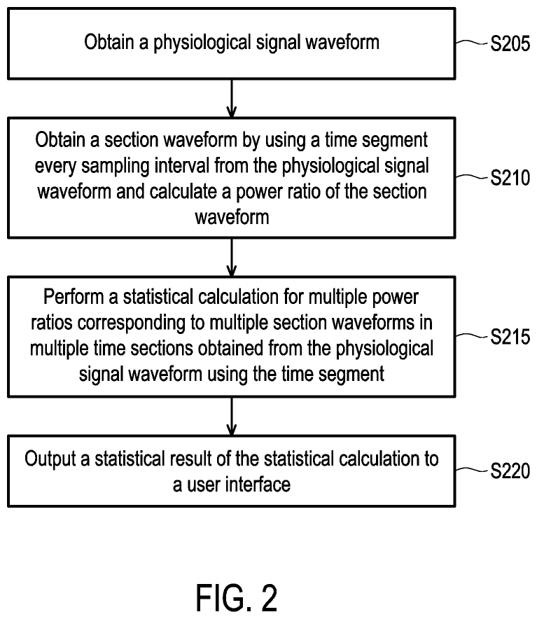

FIG. 2 is a flowchart of a method for analyzing signal waveform according to an embodiment of the disclosure. Referring to FIG. 1 and FIG. 2. In step S205, a physiological signal waveform based on a time sequence is obtained. The physiological signal waveform may be blood pressure waveform, cerebral blood flow velocity waveform (of the left cerebral artery or the right cerebral artery), cerebrovascular resistance waveform, etc. For example, the first sensor 140 measures the blood pressure wave of the person to be detected within a measurement time, and then obtains the blood pressure waveform. The cerebral blood flow velocity waveform of the left or right middle cerebral artery within a measurement time is obtained through the second sensor 150.

The cerebrovascular resistance waveform is obtained based on the blood pressure waveform and the cerebral blood flow velocity waveform. For example, multiple blood pressures and multiple cerebral blood flow velocities corresponding to multiple heartbeat cycles are obtained from the blood pressure waveform and the cerebral blood flow velocity waveform to calculate a cerebrovascular resistance value of each of the heartbeat cycles, respectively, and the cerebrovascular resistance waveform is obtained. Specifically, a mean blood pressure value corresponding to the blood pressures of each of the heartbeat cycles is calculated. As mean velocity value of the cerebral blood flow velocities corresponding to each of the heartbeat cycles is calculated. The cerebrovascular resistance value is obtained by dividing the mean blood pressure value by the mean blood flow velocity value.

In an embodiment, the time of blood pressure diastolic value is used as the starting point and end point of each of the heartbeat cycles, and the mean blood pressure value and the mean flow velocity value of each of the heartbeat cycles are calculated according to the area under respective curves of blood pressure waveform and cerebral blood flow velocity waveform. During the period of monitoring the cerebral blood flow velocity and the blood pressure, both the cerebral blood flow velocity and the blood pressure are fluctuating. The mean cerebral blood flow velocity value and the mean blood pressure value within each of the heartbeat cycles are used to calculate the cerebrovascular resistance value of each of the heartbeat cycles. Accordingly, it is known that the cerebrovascular resistance value within the period of monitoring is also fluctuating. If the cerebrovascular resistance value is high, it means that the blood flow into the great and small vessels, the capillaries, and the brain tissue decreases under a certain blood pressure. Then the spectrum analysis method of the Fourier transform is used to separate the portion representing the microcirculation of the brain tissue from the cerebral blood flow resistance value wave within this period of time, so as to obtain an amount of cerebral blood flow that may enter the small vessel and/or capillary.

Next, in step S210, a section waveform is obtained by using a time segment every sampling interval from the physiological signal waveform, and a power ratio of the section waveform is calculated. The process of calculating the power ratio of the section waveform is described below. The section waveform is converted into a spectrum. A first power sum within a first frequency range of the spectrum is calculated. A second power sum within a second frequency range of the spectrum is calculated. The first frequency range lies within the second frequency range. A ratio of the first power sum to the second power sum is calculated to obtain a corresponding power ratio.

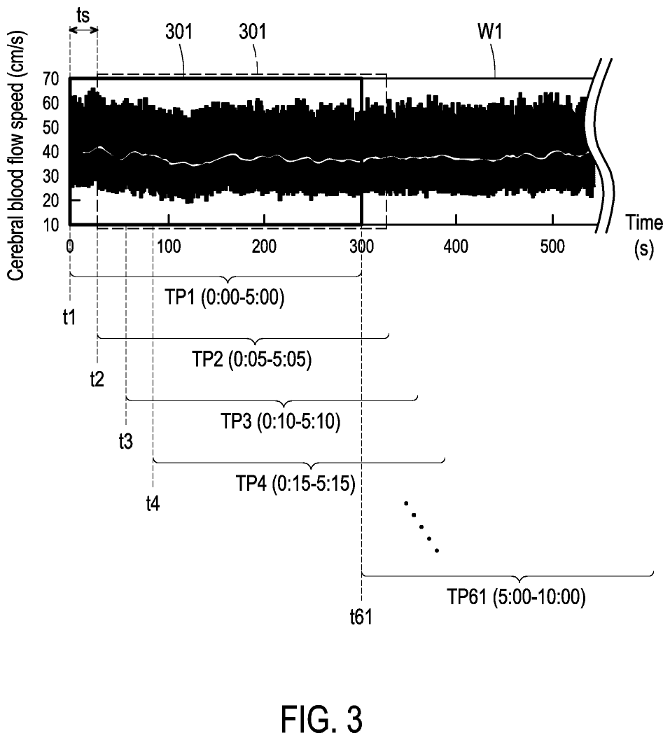

FIG. 3 is a schematic diagram of a cerebral blood flow velocity waveform according to an embodiment of the disclosure. In this embodiment, for illustration, the length of the physiological signal waveform W1 is 10 minutes, the length of the time segment 301 is 5 minutes, and the sampling interval ts is 5 seconds, however, it is not limited thereto. In this embodiment, the physiological signal waveform W1 is the cerebral blood flow velocity waveform of the left brain (the vertical axis is the cerebral blood flow velocity with a unit of cm/s). In another embodiment, the physiological signal waveform W1 is the cerebral blood flow velocity waveform of the right brain (the vertical axis is the cerebral blood flow velocity with a unit of cm/s). Alternatively, in other embodiments, the physiological signal waveform W1 may also be a cerebrovascular resistance waveform (the vertical axis is the cerebrovascular resistance with a unit of mmHg·s/cm). In addition, in another embodiment, the physiological signal waveform W1 may also be a blood pressure waveform (the vertical axis is the blood pressure with a unit of mmHg).

Specifically, first, starting from the sampling time point t1 (0:00), the time segment 301 is used to extract a section waveform TP1 of the time section 0:00 to 5:00 from the physiological signal waveform W1.

Next, 5 seconds of sampling interval ts is added to the sampling time point t1 to obtain the next sampling time point t2 (0:05), and then the time segment 301 is used to extract a section waveform TP2 of the time section 0:05 to 5:05 from the physiological signal waveform W1.

Then, 5 seconds of sampling interval ts is added to the sampling time point t2 to obtain the next sampling time point t3 (0:10), and then the time segment 301 is used to extract a section waveform TP3 of the time section 0:10 to 5:10 from the physiological signal waveform W1.

Afterwards, 5 seconds of sampling interval ts is added to the sampling time point t3 to obtain the next sampling time point t4 (0:15), and then the time segment 301 is used to extract a section waveform TP4 of the time section 0:15 to 5:15 from the physiological signal waveform W1. By analogy, 61 section waveforms TP1-TP61 may be obtained on 61 sampling points (t1-t61).

Fourier transform analysis performed for each of the section waveforms TP1-TP61 to convert the section waveforms TP1-TP61 into spectra on the frequency domain, respectively, and then the power ratio of two frequency ranges is calculated for each of the spectra. The following example illustrates how to calculate the corresponding power ratio for the spectrum corresponding to each of the section waveforms.

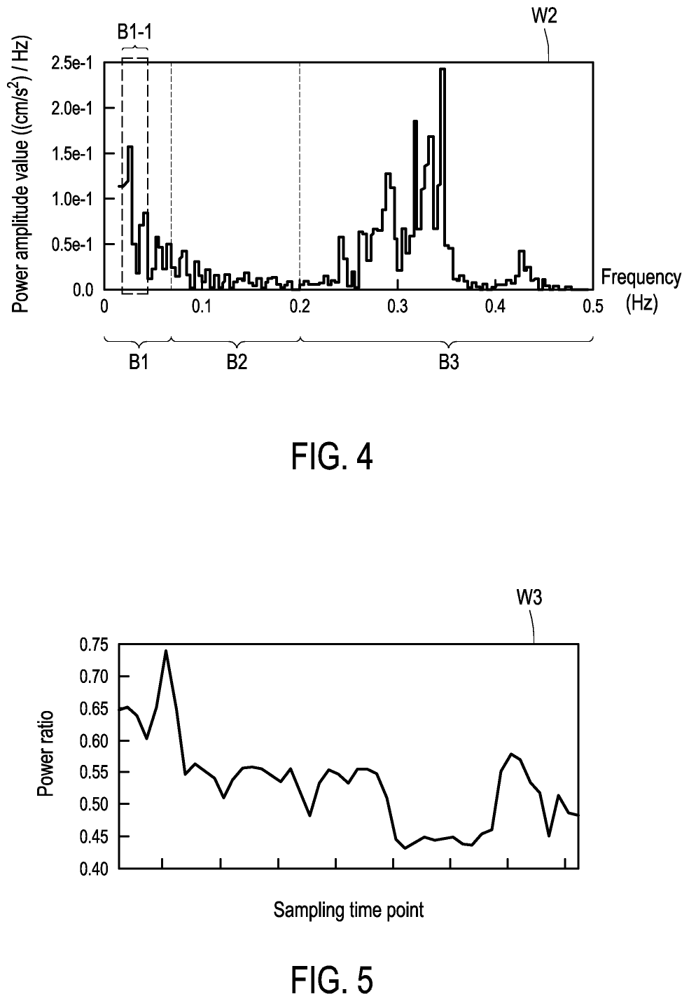

FIG. 4 is a schematic diagram of a spectrum according to an embodiment of the disclosure. In FIG. 4, the horizontal axis is the frequency, and the vertical axis is the power amplitude. In this embodiment, a spectrum W2 is divided into three frequency ranges, namely very low frequency range B1 (0.02-0.07 Hz), low frequency range B2 (0.07-0.2 Hz), and high frequency range B3 (0.2-0.5 Hz). Next, a sub-frequency range B1-1 (the first frequency range) with lower frequency is further extracted from the very low frequency range B1 (the first frequency range). The sub-frequency range B1-1 is, for example, 0.02-0.04 Hz.

Then, relative power amplitude values within 0.02-0.04 Hz (the sub-frequency range B1-1) are summed to obtain a power sum (Ps_11) of the sub-frequency range B1-1; a power sum (Ps_1) of the very low frequency range B1 is obtained by summing relative power amplitude values within 0.02-0.07 Hz. After that, the power sum of the sub-frequency range B1-1 is divided by the power sum of the very low frequency range B1 to obtain a power ratio (Ps_11/Ps_1) of the sub-frequency range B1-1 corresponding to the very low frequency range B1.

Assuming that the power sum of the spectrum corresponding to the section waveform TP1 obtained at sampling time point t1 in the sub-frequency range B1-1 (0.02-0.04 Hz) is 0.41544, and the power sum of the very low frequency range B1 is 0.72, then the power ratio=0.41544÷0.72=57.7%. The power ratio corresponding to the sampling time point t1 is 57.7%. By analogy, the power ratio corresponding to the sampling time points t1-t61 may be obtained.

In addition, in another embodiment, the very low frequency range B1 may be further divided into three sub-frequency ranges 0.02-0.04 Hz (sub-frequency range B1-1), 0.04-0.05 Hz, and 0.05-0.07 Hz. Next, relative power amplitude values within the three sub-frequency ranges of 0.02-0.04 Hz, 0.04-0.05 Hz, and 0.05-0.07 Hz are summed to obtain power sums (Ps_11, Ps_12, Ps_13) of each of the sub-frequency ranges. Then, the three power sums of the three sub-frequency ranges of 0.02-0.04 Hz, 0.04-0.05 Hz, and 0.05-0.07 Hz are summed to obtain the power sum (Ps_1=Ps_11+Ps_12+Ps_13) of the very low frequency range B1. Afterwards, the three power sums of the three sub-frequency ranges are divided by the power sum of the very low frequency range B1 respectively to obtain power ratios (Ps_11/Ps_1, Ps_12/Ps_1, Ps_13/Ps_1) corresponding to 0.02-0.04 Hz, 0.04-0.05 Hz, 0.05-0.07 Hz, respectively.

In another embodiment, power sums (Ps_1, Ps_2, Ps_3) of the very low frequency range B1 (0.02-0.07 Hz), the low frequency range B2 (0.07-0.2 Hz), and the high frequency range B3 (0.2-0.5 Hz) may be further calculated. Afterwards, the three power sums are summed to obtain the power sum (Ps_all=Ps_1+Ps_2+Ps_3) of the full frequency range total wave power of the spectrum W2. Then, the three power sums (Ps_1, Ps_2, Ps_3) are divided by the power sum (Ps_all) of the total wave power respectively to obtain power ratios (Ps_1/Ps_all, Ps_2/Ps_all, Ps_3/Ps_all) corresponding to 0.02-0.07 Hz, 0.07-0.2 Hz, 0.2-0.5 Hz, respectively.

FIG. 5 is a schematic diagram of a power ratio tendency waveform according to an embodiment of the disclosure. In FIG. 5, the horizontal axis is the sampling time point, and the vertical axis is the power ratio. That is, referring to FIG. 5, a power ratio tendency waveform W3 represents the trend of the power ratio corresponding to the sampling time points t1-t61.

Next, in step S215, a statistical calculation is performed for multiple power ratios corresponding to multiple section waveforms TP1-TP61 in multiple time sections obtained from the physiological signal waveform W1 using the time segment 301. In addition, in step S220, a statistical result of the statistical calculation is output to a user interface.

In an embodiment, a storage 120 of the electronic apparatus 100 includes a user interface, and the user interface is displayed through the display 130. After obtaining the statistical result, the statistical result may be further output to the user interface. In addition, various waveforms may also be displayed on the user interface. For example, the physiological signal waveform W1, the power ratio tendency waveform W3, etc., may be displayed on the user interface, and then the statistical result is further output on the power ratio tendency waveform W3. In addition, the frequency range used may be displayed on the user interface simultaneously. For example, the user interface may only list 0.02-0.04 Hz and the corresponding statistical result thereof. Alternatively, the user interface may list the statistical result corresponding to 0.02-0.04 Hz and 0.02-0.07 Hz. Alternatively, the user interface may lists the very low frequency range B1 (0.02-0.07 Hz), the low frequency range B2 (0.07-0.2 Hz), the high frequency range B3 (0.2-0.5 Hz), and the corresponding statistical results thereof, as well as the three sub-frequency ranges 0.02-0.04 Hz, 0.04-0.05 Hz, and 0.05-0.07 Hz included in the very low frequency range B1 (0.02-0.07 Hz) and corresponding statistical results thereof.

In other embodiments, the electronic apparatus 100 may also use wireless or wired communication technology to transmit the obtained physiological signal waveform W1, the power ratio tendency waveform W3, and other waveforms and statistical results to other electronic apparatuses (e.g., an electronic apparatus used by a specialist physician).

The statistical calculation includes at least one of the following process: calculating an average value of the power ratios; calculating a coefficient of variation (CV) of the power ratios; calculating a first quartile (Q1), a second quartile (Q2), and a third quartile (Q3) of the power ratios; and calculating an area occupied by power ratios greater than a default value in the power ratio tendency waveform W3 obtained based on the power ratios. For example, assuming that the default value is 0.5, the area occupied by the power ratio greater than 0.5 in the power ratio tendency waveform W3 is calculated.

In the field of brain signal, 0.02-0.04 Hz (the sub-frequency range B1-1) may be related the frequency of the cerebral cortex. Symptoms such as brain fog, and stroke are associated with impaired cerebral cortical microcirculation. If the cerebral cortical microcirculation is impaired, a part of the microcirculation resistance increases significantly. Thus, the power ratio occupied by the frequency range of 0.02-0.04 Hz may increase. Generally, compared with the power ratio of the very low frequency range (0.02-0.07 Hz), in response to the power ratio of the frequency range of 0.02-0.04 Hz being greater than 0.5, symptoms such as brain fog, and stroke are more likely to occur. Accordingly, setting the default value to 0.5 may give an early warning of whether the microcirculation of the cerebral cortex is impaired. The above is for illustrative purposes only and not as a limitation.

The disclosure provides a non-transitory computer-readable recording medium that is configured to store a code. In response to the code being executed by the processor 110, the processes of the method for analyzing signal waveform are executed.

To sum up, the disclosure analyzes the power ratios corresponding to a specified frequency range in the section waveforms of the physiological signal, and performs statistical calculation on the power ratios, then outputs the statistical result to the user interface, so that the user may more intuitively determine whether there is an abnormal risk in a person to be detected.

What is claimed is:

1. A method for analyzing signal waveform, which is executed by a processor, and the method comprising:
obtaining a physiological signal waveform based on a time sequence;
obtaining a section waveform by using a time segment every sampling interval from the physiological signal waveform, and calculating a power ratio of the section waveform, wherein calculating the power ratio of the section waveform comprises:
converting the section waveform into a spectrum;
calculating a first power sum within a first frequency range of the spectrum;
calculating a second power sum within a second frequency range of the spectrum, wherein the first frequency range lies within the second frequency range; and
calculating a ratio of the first power sum to the second power sum to obtain the power ratio;
performing a statistical calculation for a plurality of power ratios corresponding to a plurality of section waveforms in a plurality of time sections obtained from the physiological signal waveform using the time segment; and
outputting a statistical result of the statistical calculation to a user interface.

2. The method for analyzing signal waveform according to claim 1, wherein the physiological signal waveform is a cerebrovascular resistance waveform, and the method further comprises:
obtaining a blood pressure waveform within a measurement time through a first sensor;
obtaining a cerebral blood flow velocity waveform within the measurement time through a second sensor; and
obtaining a plurality of blood pressures and a plurality of cerebral blood flow velocities corresponding to a plurality of heartbeat cycles from the blood pressure waveform and the cerebral blood flow velocity waveform to calculate a cerebrovascular resistance value of each of the heartbeat cycles, respectively, and obtaining the cerebrovascular resistance waveform.

3. The method for analyzing signal waveform according to claim 2, wherein calculating the cerebrovascular resistance value of each of the heartbeat cycles comprises:
calculating a blood pressure average value corresponding to the blood pressures of each of the heartbeat cycles;
calculating a speed average value of the cerebral blood flow velocities corresponding to each of the heartbeat cycles; and
dividing the blood pressure average value by the speed average value to obtain the cerebrovascular resistance value.

4. The method for analyzing signal waveform according to claim 2, wherein the first sensor is a blood pressure monitor, and the second sensor is a transcranial Doppler ultrasonic device.

5. The method for analyzing signal waveform according to claim 1, wherein the physiological signal waveform is a cerebral blood flow velocity waveform, and the method further comprises:
obtaining the cerebral blood flow velocity waveform within a measurement time through a transcranial Doppler ultrasonic device.

6. The method for analyzing signal waveform according to claim 1, wherein the physiological signal waveform is a blood pressure waveform, and the method further comprises:
obtaining the blood pressure waveform within a measurement time through a blood pressure monitor.

7. The method for analyzing signal waveform according to claim 1, wherein the statistical calculation comprises at least one of the following:
calculating an average value of the power ratios;
calculating a coefficient of variation of the power ratios;
calculating a first quartile, a second quartile, and a third quartile of the power ratios; and
calculating an area occupied by power ratios greater than a default value in a power ratio tendency waveform obtained based on the power ratios.

8. The method for analyzing signal waveform according to claim 1, wherein the first frequency range is 0.02-0.04 Hz, and the second frequency range is 0.02-0.07 Hz.

9. An electronic apparatus, comprising:
a storage, storing at least one code segment; and
a processor, coupled to the storage, and configured to execute the at least one code segment for:
obtaining a physiological signal waveform based on a time sequence;
obtaining a section waveform by using a time segment every sampling interval from the physiological signal waveform, and calculating a power ratio of the section waveform, wherein calculating the power ratio of the section waveform comprises:
converting the section waveform into a spectrum;
calculating a first power sum within a first frequency range of the spectrum;
calculating a second power sum within a second frequency range of the spectrum, wherein the first frequency range lies within the second frequency range; and
calculating a ratio of the first power sum to the second power sum to obtain the power ratio;
performing a statistical calculation for a plurality of power ratios corresponding to a plurality of section waveforms in a plurality of time sections obtained from the physiological signal waveform using the time segment; and
outputting a statistical result of the statistical calculation to a user interface.

10. A non-transitory computer-readable recording medium configured to store a code, wherein in response to the code being executed by a processor, the processor is made to:
obtain a physiological signal waveform based on a time sequence;
obtain a section waveform by using a time segment every sampling interval from the physiological signal waveform, and calculate a power ratio of the section waveform, wherein calculating the power ratio of the section waveform comprises:
converting the section waveform into a spectrum;
calculating a first power sum within a first frequency range of the spectrum;
calculating a second power sum within a second frequency range of the spectrum, wherein the first frequency range lies within the second frequency range; and
calculating a ratio of the first power sum to the second power sum to obtain the power ratio;
perform a statistical calculation for a plurality of power ratios corresponding to a plurality of section waveforms in a plurality of time sections obtained from the physiological signal waveform using the time segment; and
output a statistical result of the statistical calculation to a user interface.
